(19) United States
(12) Patent Application Publication
(10) Pub. No.: US 2007/0160634 A1
(43) Pub. Date: Jul. 12, 2007

(54) Methods for reducing somatic cell count in milk

(75) Inventor: Gregory I. Bohach, Moscow, ID (US)

Correspondence Address: KLARQUIST SPARKMAN, LLP, 121 SW SALMON STREET, SUITE 1600, PORTLAND, OR, 97204, US

(73) Assignee: Idaho Research Foundation, Inc., Moscow, ID (US)

(21) Appl. No.: 10/585,554

(22) PCT Filed: Jan. 7, 2005
(86) PCT No.: PCT/US05/00482

§ 371 Date: Mar. 19, 2007

Related U.S. Application Data
(60) Provisional application No. 60/535,454, filed on Jan. 8, 2004.

(51) Int. Cl. A61K 39/085

(52) U.S. Cl. 424/243.1

(57) ABSTRACT

In an embodiment, the present invention is directed to a method of reducing the somatic cell count in milk including administering an effective amount of a toxin to a mammal. In some embodiments, the toxin administered has a modified disulfide loop region. For example, at least 40% of the amino acid residues within the disulfide loop region are deleted in some embodiments. The mammal reacts to the administered toxin with an immune response. In an embodiment, the present invention is directed a method of increasing the quality of milk produced by mammals including administering an effective amount of a toxin to a mammal.

### FIELD OF THE INVENTION

The present invention relates to methods for reducing somatic cell count in milk. More particularly, the invention is directed to methods of administering compositions capable of causing an immune response in milk-producing mammals, which leads to a reduced somatic cell count in the milk produced.

### BACKGROUND OF THE INVENTION

The somatic cell count (SCC) of milk is commonly used as a measure of milk quality. Somatic cells are simply animal body cells present at low levels in normal milk. However, high levels of these cells in milk can indicate abnormal, reduced-quality milk that is usually associated with intramammary bacterial infection (mastitis).

Milk markets routinely rely on somatic cell counts to help ensure a quality product. Somatic cell count levels are monitored to assure compliance with state and federal milk quality standards. Because most markets pay a premium for low SCC, good-quality milk, reducing somatic cell count levels can result in greater revenues for milk producers.

Therefore, a need exists for methods for reducing the somatic cell count in milk.

### SUMMARY OF THE INVENTION

In an embodiment, the present invention is directed to a method of reducing the somatic cell count in milk including administering an effective amount of a toxin to a mammal. In some embodiments, the toxin administered has a modified disulfide loop region. For example, at least 40% of the amino acid residues within the disulfide loop region are deleted in some embodiments. The mammal reacts to the administered toxin with an immune response. In an embodiment, the present invention is directed a method of increasing the quality of milk produced by mammals including administering an effective amount of a toxin to a mammal.

### DRAWINGS

The invention may be more completely understood in connection with the following drawings, in which:

FIG. 1A shows the change in somatic cell count after SEC1 treatment (1 mg per quarter) wherein the SEC1 is delivered by intramammary delivery with surgically implanted osmotic pumps.

FIG. 1B shows the change in somatic cell count after SEC1 treatment (1 mg per quarter) wherein the SEC1 is delivered by infusion through the teat canals.

While the invention is susceptible to various modifications and alternative forms, specifics thereof have been shown by way of example and drawings, and will be described in detail. It should be understood, however, that the invention is not limited to the particular embodiments described. On the contrary, the intention is to cover modifications, equivalents, and alternatives falling within the spirit and scope of the invention.

### DETAILED DESCRIPTION OF THE INVENTION

Somatic cell count is used as an indication of mastitis, an infection of the mammary gland. In diary cows, the primary cause of infection is bacteria. The major contagious pathogens are Streptococcus agalactiae, Staphylococcus aureus, and Mycoplasma species. S. aureus is considered the most common source of contagious mastitis. The bacteria damage the duct system and establish deep-seated pockets of infection in the milk secreting tissues followed by abscess formation and walling-off of bacteria by scar tissue. This walling-off phenomena is partially responsible for the poor cure rates of S. aureus infections by antibiotic therapy.

Staphylococcal enterotoxins are superantigens (SAgs), proteins capable of stimulating the immune system by binding to conserved parts of T cell receptor (TCR) and molecules of the major histocompatibility complex class II (MHCII). SAgs bypass antigenic specificity of the TCR and are extremely potent stimulators of T cells, effective at picomolar concentrations. Unfortunately the excessive and aberrant immune cell stimulation has serious deleterious effects including induction of shock, hypotension, T cell apoptosis and eventual immunosuppression. Staphylococcal enterotoxins also have emetic properties by which they have the ability to cause food poisoning.

The production of staphylococcal enterotoxin C (SEC) or other similar acting toxins is believed to enable S. aureus bacteria to persistently colonize the mammary gland by inhibition of immune responses capable of pathogen clearance.

In an embodiment, the present invention is directed to a method for reducing the somatic cell count in milk comprising administering to a mammal an effective amount of a composition comprising a toxin. The mammal responds to the administered toxin by generating an immune response. The immune response of the animal is such that it reduces the somatic cell count in milk. The present invention also includes a method of increasing the quality of milk produced by mammals.

Compositions Administered

In some embodiments, the toxin administered in the method of the invention may be a wild-type toxin. In other embodiments, the toxin administered may be a mutant toxin that retains useful biological properties but has a substantially reduced level of toxicity.

In an embodiment, the toxin is a modified pyrogenic toxin. The pyrogenic toxins constitute a family of exotoxins produced by species of gram positive cocci, such as Staphylococcusand Streptococcus. The pyrogenic toxins are characterized by shared ability to induce fever, enhance host susceptibility to endotoxin shock, and induce T cell proliferation through action as superantigens. Examples of pyrogenic toxins include TSST-1, staphylococcal enterotoxins (SEs), and streptococcal pyrogenic exotoxins (SPEs). In addition to the activities listed above, some pyrogenic toxins have additional activities that are not shared by all pyrogenic toxins. For example, the staphylococcal enterotoxins induce emesis and diarrhea when ingested. Structurally, the pyrogenic toxins have varying degrees of relatedness at the amino acid and nucleotide sequence levels. However, a number of the pyrogenic toxins include a disulfide loop as a structural feature. The staphylococcal enterotoxins have a disulfide loop, as do some others in this family. Examples of other pyrogenic toxins that have a disulfide loop are the streptococcal superantigen (“SSA”) and streptococcal pyrogenic exotoxin A (“SPEA”).

In some embodiments, the mutant toxin used is a modified version of a disulfide loop containing bacterial pyrogenic toxin. Such modified pyrogenic toxins retain useful biological properties but have substantially reduced toxicity (e.g., toxicity reduced by at least about 10-fold) compared to the corresponding unmodified native toxin. Modified toxins used in accordance with the invention may also have reduced emetic properties and/or reduced pyrogenicity in comparison with the unmodified wild-type toxins.

In some embodiments, the modified pyrogenic toxins are derived from a native pyrogenic toxin having a disulfide loop. The terms “disulfide loop” and “disulfide loop region” are used interchangeably herein. As employed in this application, these terms refer to the sequence of about 10 to about 30 amino acid residues forcing a loop defined by a disulfide bond in a native pyrogenic toxin. The term “disulfide loop region” also refers to the corresponding portion of the sequence of a modified pyrogenic toxin which has been produced by deletion, substitution or addition of one or more amino acid residues of the disulfide loop of a native pyrogenic toxin. The disulfide loop region is defined to begin with the N-terminal Cys residue and end with the C-terminal Cys residue of the loop, e.g.,. amino acid residues 93-110 of staphylococcal enterotoxin C-1 (SEC1). Table 6 shows the alignment of the predicted sequences of the eight known SEC variants following cleavage of the signal peptide. The N-terminus of each of the mature proteins was verified by amino acid sequencing. As used herein, the positions of the disulfide loop region for a given native pyrogenic toxin are numbered beginning with the N-terminal cyteine residue in the loop, e.g., position 93 of type B or C staphylococcal enterotoxins is also referred to herein as position 1 of the disulfide loop region.

In some embodiments, the modification of the disulfide loop typically includes deletion of at least about 40% of the amino acid residues within the disulfide loop. For example, this generally results in the deletion of at least about 8 amino acid residues from the disulfide loop region of an SEC.

Examples of native staphylococcal enterotoxins which can be modified to form the present low toxicity toxins include type A, B, C₁, C₂, C₃, D, E, G, and H staphylococcal enterotoxins. The toxin used with the present invention may also be an antigenic portion of a staphylococcal enterotoxin. A family of mutant SEC toxins with reduced toxicity has been developed. Examples of such mutants include those disclosed in PCT filing PCT/US98/25107 (Pub. # WO9927889) and U.S. application Ser. No. 09/555,115 (NON-TOXIC IMMUNE STIMULATING ENTEROTOXIN COMPOSITIONS) the disclosures of which are herein incorporated by reference. Examples of such mutants are also shown in Table 5 below.

Examples of mutant toxins used with the invention further include disulfide loop region deletion mutants of native toxins derived from Streptococcus pyrogenes. For example, suitable native disulfide loop-containing toxins which may be modified according to the present invention include streptococcal pyrogenic enterotoxin A (“SPEA”) and streptococcal superantigen (“SSA”) produced by S. pyrogenes.

The mutant used in an embodiment can be produced by deleting amino acid residues 95-106 (disulfide loop residues 3-14) of staphylococcal enterotoxin C-1. This mutant is known as SEC1-12. One of skill in the art will appreciate that in some embodiments any SEC toxin that is capable of causing an immune response that is cross-reactive with wild type SEC toxin but with reduced toxicity may be used. In some embodiments, the composition used in the method of the invention contains more than one type of mutant toxin, and therefore in those embodiments the composition is made up of mixtures of different types of mutant toxins.

In some embodiments, the composition that is administered may contain other bacterial components besides an enterotoxin. In other embodiments, the composition does not contain any other bacterially derived components besides an isolated mutant enterotoxin. Bacterially derived components, as used in the application, refers to those components which can be found in or generated from a bacterial organism, including heterogeneous mixtures of cellular material, proteins of all types, polysaccharides, etc. Accordingly, in an embodiment, the composition used in the method of the invention comprises a mutant toxin but does not comprise any other bacterial or bacterially derived components.

The compositions used with the present invention may be suspended in liquid medium for infusion or injection according to known protocols. Any appropriate carriers, diluents, buffers, stabilizers, and adjuvants known in the art may be used. Suitable suspension liquids include saline solution, water, and physiologic buffers.

In some embodiments, the use of adjuvants is desirable. Suitable adjuvants for use in the compositions of the invention include Freund's complete adjuvant (FCA), Freund's incomplete adjuvant (FIC), adjuvant 65, cholera toxin B subunit, aluminum hydroxide A1(OH)₃; or Bordetella pertussis, muramyl dipeptide, cytokines and saponin.

In an embodiment, the invention includes the use of a modified toxin in the manufacture of a medicament for reducing somatic cell count in the milk of non-human mammals.

Administration of Compositions

The compositions used in the methods of the invention can be administered via enteral or parenteral routes such as oral, intranasal, intravenous, intraperitoneal, intramuscular, subcutaneous, intradermal, intramammary, intraruminal, or other suitable routes.

Sites for intramuscular administration may include the left and right sides of the brachio chepalic muscle. In some embodiments, the composition may be administered behind, or on, the ear of the mammal. The precise site of administration for any route of administration may, of course, vary according to known administration protocols. The amount and form of the composition administered will also vary according to the formulation used and the mammal to be immunized. In some embodiments the amount administered is an amount effective to cause an immune response in the subject lactating mammal such that the somatic cell count is reduced.

Generally, the antigen is injected using a syringe and needle for intramuscular and intraperitoneal routes and fine-bore polyethylene surgical tubing fitted to a syringe for the intramammary route or alternatively a conventional sterile intramammary applicator. For intramammary administration, the composition may be administered via the major lactiferous duct or the supramammary lymph node. In some embodiments the administration is via the teat orifice into the teat canal. In an embodiment, the composition may be delivered with surgically implanted osmotic pumps.

In some embodiments, a single dose of the composition is administered. In other embodiments, a single dose is administered followed by booster doses administered periodically. The time for administration of the toxin may vary. In some embodiments the time may be prior to a predicted day of parturition for a mammal. In other embodiments, the time may be after a predicted day of parturition for a mammal.

The amount of the composition administered may vary depending on various factors including the method of administration, the existing somatic cell count, the time period of administration, etc. The amount administered may be an effective amount to reduce the somatic cell count in milk. In some embodiments, less than 0.1 μg/kg of body weight of the toxin may be administered. In other embodiments, the amount may range from 0.1-10.0 μg/kg of body weight. In still other embodiments, the amount may be greater than 10.0 μg/kg of body weight. In an embodiment, about 0.1 mg to about 10.0 mg of toxin is administered. Alternatively, about 4.0 mg of toxin may be administered.

In some embodiments, a live S. aureus organism that expresses a mutant toxin may be administered to the subject mammal.

Populations Treated

Mammals that may be treated according to the invention include Bos taurus. In some embodiments, the method of the present invention may be used to treat an entire herd of milk producing mammal. In other embodiments, only those mammals with elevated somatic cell counts are treated in accordance with the methods of the invention. The average somatic cell count among dairy cattle has been disclosed as 257,000 cells per ml nationally (Bulk Tank Milk Somatic Cell Counts and Your Milk Quality Assurance Program, USDA, (January 1994)). Therefore, in some embodiments those individual mammals with somatic cell counts above 257,000, or above average, are treated. In other embodiments, if the average somatic cell count among a particular herd is above the national average, then the whole herd is treated.

The following examples further illustrate the present invention. They are in no way to be construed as a limitation in scope and meaning of the claims.

### EXAMPLES

### Example 1

### Effect of Toxin on Somatic Cell Counts

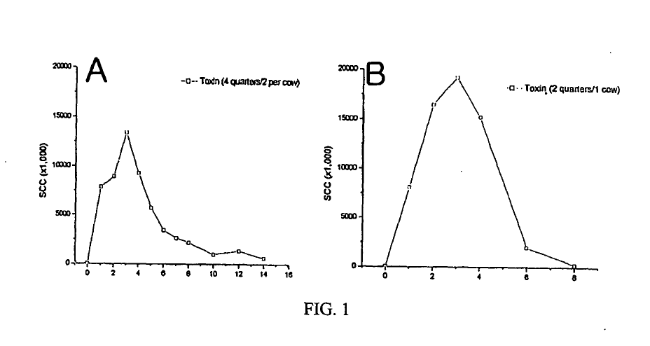

Mammary glands of cows were infused with a solution containing staphylococcal enterotoxin C (SEC), but no S. aureus bacteria. Within one day, the somatic cell count increased by about 7,500,000 cells. See FIG. 1A-B. Therefore, the presence of toxin alone greatly increases the production and/or release of somatic cells into the milk.

### Example 2

### Lethality of Toxins in Rabbit and Monkey Models

SEC1-12 was found to be one-to two orders of magnitude less toxic than SEC1. For example, although as little as 0.1 μg/kg of body weight of SEC1 resulted in shock and death in rabbit toxin shock model, even 100 μg/kg of SEC1-12 failed to induce lethality in this model (Table 1). Moreover, SEC1-12 failed to induce emesis in monkeys. Furthermore, SEC1-12 was only minimally pyrogenic.

TABLE 1
Toxicity of SEC1 and SEC1-12

| | Emesis¹ | | Endotoxin lethality² | | Pyrogenicity³ | |
|---|---|---|---|---|---|---|
| | SEC1 | SEC1-12 | SEC1 | SEC1-12 | SEC1 | SEC1-12 |
| 250 μg/kg⁴ | — | 0/2 | — | — | — | — |
| 100 μg/kg | — | 0/2 | — | 0/3 | — | 0.6 |
| 10 μg/kg | 3/3 | — | 3/3 | 0/3 | 1.6 | 0.45 |
| 1 μg/kg | 3/3 | — | 2/2 | — | 1.3 | — |
| 0.1 μg/kg | 2/2 | — | 3/3 | — | 1.05 | — |
| 0.01 μg/kg | 0/2 | — | 1/4 | — | 0.475 | — |

¹Number of monkeys (Macaca nemestrina) exhibiting emesis/number of animals challenged.
²Tested in rabbit model of toxic shock; number of animals dying/number of animals challenged.
³The mean maximum rectal temperature rise (° C.) following intravenous administration of enterotoxin.
⁴Dose of toxin per kg of body weight.
Source: Callantine, 1997.

### Example 3

### Lethality of Toxins in Bovine Model

Increasing doses of SEC1-12 were injected subcutaneously (in the supramammary lymph node area) or infused into the mammary gland, and cows were observed for physiological reactions indicating symptoms associated with a shock response. In an initial preliminary toxicity study, one cow was injected with 0.1 mg of SEC1-12 (<0.2 μg/kg of body weight). This animal did not show any noticeable changes in respiration rate or temperature. Subsequently, 5 cows were infused or injected with larger doses of SEC1-12 raging from 0.5 to 2.0 mg (1.0-4.0 μg/kg) and examined initially twice daily for 1-2 days and than once per day for a total of 3-6 days (Table II). The following parameters were recorded: heart rate, respiratory rate, rumen contractions rate, and temperature. The cows treated with SEC1-12 exhibited a transient increase in temperature, followed in all examined cases by a drop of temperature below an initial value (Table II), apparently resulting from a physiological overcompensation. The increase in body temperature appeared to be greater in cows receiving injection of toxin (2.2±1.7 F, n=2) (mean±SD), compared to cows receiving infusion into the mammary gland (0.4±0.3 F, n=3). A small increase in body temperature was also observed in prior experiments with rabbits (Table I) and is consistent with a premise of IL-1 (a potent intrinsic pyrogen) release, likely to be induced by SEC1-12. The appearance of orifices, eyes, tongue, posture, salivation, and behavior remained normal in all cows treated with SEC1-12.

TABLE 2
SEC1-12 safety studies.

| Cow | | Dates of | Temp. | Δ temp. | Respiration | Heart | Rumen |
|---|---|---|---|---|---|---|---|
| # | Treatment¹ | exams² | (° F.) | (° F.)³ | rate/min | rate/min | contr./min. |
| 514 | PBS | 1.25.00 | 100.5 | — | 30 | 100-110 | 2 |
| | infusion | 1.26.00 | 100.4 | −0.1 | 30 | 70 | 2 |
| | (control) | 1.27.00 | 101.1 | +0.6 | 30 | 60 | ND⁴ |
| | | 1.28.00 | 101.2 | +0.7 | 30 | 50 | ND |
| | | | 100.2 | −0.3 | 40 | 80 | ND |
| 673 | PBS | 4.04.00 | 102.5 | — | 40 | 60 | 1 |
| | injection | 4.06.00 | 102.3 | −0.2 | 40 | 60 | ND |
| | (control) | 4.10.00 | 101.0 | −1.5 | 45 | 60 | ND |
| 675 | SEC1-12 | 4.04.00 | 101.6 | — | 30 | 60 | 2 |
| | injection | 4.06.00 | 102.6 | +1.0 | 30 | 60 | ND |
| | (2.0 mg) | 4.10.00 | 100.8 | −0.8 | 30 | 60 | ND |
| 348 | SEC1-12 | 3.21.00 | 101.0 | — | 20 | 60 | 1 |
| | injection | | 104.4 | +3.4 | ND | ND | ND |
| | (1.0 mg) | 3.22.00 | 99.3 | −1.7 | 30 | 50 | ND |
| | | | 100.7 | −0.3 | 35 | 50 | ND |
| | | 3.23.00 | 101.5 | +0.4 | 40 | 60 | |
| | | 3.24.00 | 101.6 | +0.5 | 50 | 100 | ND |
| 656 | SEC1-12 | 1.25.00 | 98.8 | — | 40 | 60-70 | 1-2 |
| | infusion | 1.26.00 | 99.0 | +0.2 | 30 | 70 | 1 |
| | (1.0 mg) | 1.27.00 | 98.3 | −0.5 | 30 | 70 | ND |
| | | 1.28.00 | 98.5 | −0.3 | 30 | 70 | ND |
| | | 1.28.00 | 99.8 | 0.0 | 30 | 60 | ND |
| 514 | SEC1-12 | 2.01.00 | 99.6 | — | 40 | 70 | 2 |
| | infusion | | 101.4 | +0.8 | 30 | 70 | |
| | (0.5 mg) | 2.02.00 | 99.2 | −0.4 | 35 | 70 | 2 |
| | | | 99.1 | −0.5 | 30 | 70 | |
| | | 2.03.00 | 99.8 | +0.2 | 40 | 70 | 1-2 |
| | | 2.04.00 | 99.7 | +0.1 | 30 | 60 | ND |
| | | | 100.4 | +0.8 | 40 | 60 | ND |
| 429 | SEC1-12 | 3.21.00 | 101.5 | — | ND | 60 | ND |
| | infusion | | 101.8 | +0.3 | ND | ND | 1 |
| | (1.0 mg) | 3.22.00 | 100.3 | −1.2 | 30 | 60 | ND |
| | | | 101.3 | −0.2 | 30 | 60 | ND |
| | | 3.23.00 | 100.7 | −0.9 | 30 | 60 | ND |
| | | 3.24.00 | 100.7 | −0.9 | 30 | 50 | ND |
| | | 3.25.00 | 97.5 | −4.0 | 30 | 50 | |

¹SEC1-12 was infused in 50 ml PBS into one quarter, or injected in 10 ml PBS into supramammary lymph node area. Control cows were treated by infusion or injection of PBS.
²Exams performed on the same day were done 4 to 6 hr apart. Cows were examined prior to treatment.
³temperature difference in relation to the initial pre-treatment temperature
⁴not determined; weak or slow contractions, difficult to detect.

### Example 4

### Effect of Toxin on Premature Bovine Delivery

As staphylococcal enterotoxin can have various negative effects in vivo, it was thought that administration of such a toxin may detrimentally cause premature delivery when administered to dairy cattle. To address this concern, the differences between the expected and actual times of delivery were compared among groups of cows treated with toxin within the last four weeks before predicted day of parturition and cows not treated. The dates of deliveries were predicted by adding 290 days to each animal date of successful insemination.

Cows treated by SEC1-12 injection calved 5.8±2.2 days before a predicted parturition day (mean±SD, n=8). Cows treated by single infusion of SEC1-12 into the mammary gland calved 2.5±0.7 days earlier than expected (n=3), whereas cows treated by two infusions calved 2.4±3.0 days earlier than expected (n=5). Untreated cows calved 2.3±2.8 days earlier than expected (n=5). The differences between groups were not statistically significant. Thus, surprisingly, it was found that infusion of SEC1-12 into the mammary gland in the last month of pregnancy does not induce premature delivery.

### Example 5

### Immunomodulatory Activity of SEC

Exposure of animals to native superantigens (SAgs) may result in an anergy or deletion of reactive T lymphocytes. Thus, the effects of SEC1-12 on reactive T cells exposed in vivo were determined.

Injection of 1.0 mg of SEC1-12 had a systemic effect on the immune system of recipient cows. PBMC (peripheral blood mononuclear cells) obtained 1-3 weeks after this treatment and cultured with SEC1 or SEC1-12 used at graded concentrations exhibited greatly increased proliferation in response to the toxins. The increase in proliferative response was much greater in a cow treated with SEC1-12 than in a control cow injected with PBS. In the latter case, the observed small but significant increase was probably related to an increase in proliferative responses of lymphocytes that commonly occurs after parturition.

These results show that SEC1-12 does not induce anergy or deletion of the reactive T lymphocytes in vivo, since the PBMC sampled after injection were capable of vigorous proliferation in response to stimulation with SEC1-12. Moreover, these results demonstrate that T lymphocytes responding to SEC1-12 participate in lymphocyte traffic and are present in systemic circulation.

### Example 6

### Immunization in a Rabbit Model

Rabbit experiments demonstrated that animals immunized with SEC1-12 were protected from SEC1. A standard toxic shock model screening was performed. The rabbits were first immunized with SEC1-12 at a dose of 25-50 μg (administered with Freund's incomplete adjuvant). Immunizations were continued until hyper-immune serum antibodies were detected by double immunodiffusion assays. Seven days after the final injection, the rabbits were challenged with an intravenous injection of native SEC1(15 μg/kg) and LPS as in the standard assay. The results (Table 3) demonstrate that immunization with SEC1-12 protected the rabbits against the effect of the native SEC1.

TABLE 3
Protection of rabbits immunized with SEC1-12 mutant against SEC1^{a}

| | No. of dead animals/total no. animals |
|---|---|
| Immunizing toxin | when challenged with SEC1^{b} |
| SEC1-12 | 0/5 |
| None | 4/5 |

^{a}Animals were challenged with SEC1 followed by intravenous endotoxin dose (10 μg/kg) Survival indicates immunity to the enhancement of lethal endotoxic shock by SEC1
^{b}15 μg of biologically active SEC1 for each kg of animal body weight.

### Example 7

### Cross-reactivity of Antibodies to SEC1-12

It has also been demonstrated that native SEC1 and SEC1-12 are antigenically identical by immunodiffusion assay. Thus, antibodies to SEC1-12 respond to SEC1 in the same way as they respond to SEC14-12. Therefore, dairy cows respond to immunization with SEC1-12 by producing antibodies that neutralize the effects of the native SEC1 toxin.

### Example 8

### Reduction of Somatic Cell Count in Lactating Dairy Cows

SEC1-12 was administered to three different groups of animals over a period of time and the somatic cells counts from each group were monitored. Group A included 12 lactating dairy cows that had an average somatic cell count of 341,700 before treatment began. Group B included 13 lactating dairy cows that had an average somatic cell count of 998,500 before treatment began. Group C included 14 lactating dairy cows that had an average somatic cell count of 650,000 before treatment-began. 4.0 mg of SEC1-12 was administered by injection to each of the animals in week 0, week 2, and week 6. The somatic cell count for each of the animals was measured 7 days after the first treatment, 7 days after the second treatment, 7 days after the third treatment, and approximately 3 months after the third treatment. The results are summarized below in Table 4 below.

TABLE 4

| | | Somatic cell count (×1,000/ml) | | | | |
|---|---|---|---|---|---|---|
| | No. | | | | | 3 |
| | of | Before | 7 days | 7 days | 7 days | months |
| | Ani- | injec- | after 1^{st} | after 2^{nd} | after 3^{rd} | after 3^{rd} |
| Group | mals | tion | injection | injection | injection | injection |
| A | 12 | 341.7 | 256.6 | 543.6 | 213.6 | 213.2 |
| B | 13 | 998.5 | 396.1 | 671.1 | 472.7 | 578.1 |
| C | 14 | 650.0 | 771.0 | 817.3 | 429.5 | 346.8 |
| Total | 39 | 677.8 | 492.3 | 687.1 | 380.7 | 386.1 |

The slight increase in somatic cell count observed seven days after the second injection is consistent with the time frame of the initial immune reaction in the animals that is induced by the mutant toxin. This slight increase is followed by a sharp decrease in somatic cell count as measured seven days after the third injection.

Specifically, the data show that the somatic cell count was reduced from an average of 677,800 per animal before treatment with SEC1-12 to approximately 380,700 per animal as measured seven days after the third injection with SEC1-12 . This corresponds to a reduction in the somatic cell count of approximately 44%. Accordingly, this example demonstrates that the somatic cell count in milk can be reduced, and therefore the quality of milk produced can be increased, through the administration of a mutant toxin, such as SEC1-12.

The data further shows that when the somatic cell count was measured three months after the third injection, that the somatic cell count was still only 386,100 on average, or approximately 43% less than before treatment began. Therefore, this example demonstrates that the reduction in somatic cell count can be a last effect of the treatment with a mutant toxin, such as SEC1-12.

TABLE 5
SEC1 LOOP MUTANTS

AMINO ACID#

93 94 95 96 97 98 99 100 101 102 103 104 105 106 107 108 109 110
SEC1 (wild type)

| | | |
|---|---|---|
| AMINO ACID | Cys Tyr Phe Ser Ser Lys Asp Asn Val Gly Lys Val Thr Gly Gly Lys Thr Cys | (SEQ ID NO:1) |
| NUCLEIC ACID | TGC TAT TTT TCA TCC AAA GAT AAT GTA GGT AAA GTT ACA GGT GGC AAA ACT TGT | (SEQ ID NO:2) |
| SEC1 Loop Deletion | | |
| Mutants | | |
| −4 A.A. MUTANT | Cys Tyr Phe Ser Ser Lys Asp Asn Ala Gly Gly Lys Thr Cys | (SEQ ID NO:3) |
| | TGC TAT TTT TCA TCC AAA GAT AAT GCA --------- GGT GGC AAA ACT TGT | (SEQ ID NO:4) |
| −9 A.A. MUTANT | Cys Tyr Phe Ser Ser Gly Lys Thr Cys | (SEQ ID NO:5) |
| | TGC TAT TTT TCA TCC ------------------- GGC AAA ACT TGT | (SEQ ID NO:6) |
| −12 A.A MUTANT | Cys Cys Gly Lys Thr Cys | (SEQ ID NO:7) |
| | TGC T-- ------------------------ GT GGC AAA ACT TGT | (SEQ ID NO:8) |

TABLE 6
Amino Acid Sequence of Selected Staphylococcal Enterotoxins

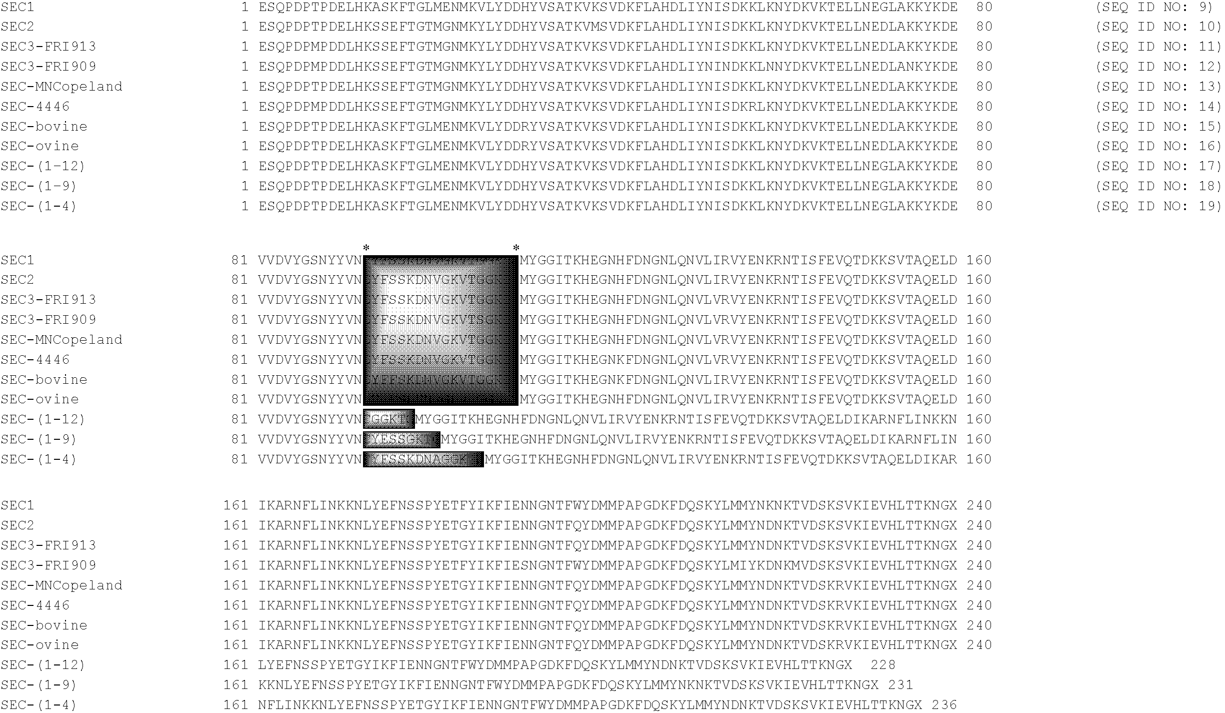

1. A method for reducing the somatic cell count in milk, comprising administering to a mammal an effective amount of a composition comprising a toxin.

2. A method for increasing the quality of milk produced by mammals, comprising administering to a mammal an effective amount of a composition comprising a toxin.

3. The method of claim 1, wherein the mammal has a somatic cell count of greater than 257,000 per ml of milk before administration of the toxin.

4. The method of claim 1, wherein the mammal is Bos taurus.

5. The method of claim 1, wherein the toxin is a staphylococcal toxin or a streptococcal toxin.

6. The method of claim 5, wherein the streptococcal toxin is streptococcal pyrogenic exotoxin A or streptococcal superantigen.

7. The method of claim 1, wherein the toxin is a type A, B, C, D, E, G, or H staphylococcal enterotoxin.

8. The method of claim 7, wherein the toxin is staphylococcal enterotoxin C (SEC).

9. The method of claim 1, wherein the toxin is a mutant toxin.

10. The method of claim 9, wherein the toxin is mutant staphylococcal enterotoxin C1-12 (SEC1-12) (SEQ ID NO: 17).

11. The method of claim 9, wherein the mutant toxin has reduced lethality, reduced emetic properties, or reduced pyrogenicity as compared with the wild-type toxin.

12. The method of claim 9, wherein the toxin has a modified disulfide loop region.

13. The method of claim 12, wherein at least 40% of the amino acid residues within the disulfide loop region are deleted.

14. The method of claim 5, wherein the toxin is an antigenic portion of a staphylococcal enterotoxin.

15. The method of claim 1, wherein about 0.1 mg to about 10.0 mg of the composition is administered.

16. The method of claim 15, wherein about 4.0 mg of the composition is administered.

17. The method of claim 1, wherein a plurality of doses of a composition comprising a toxin is administered.


SEQUENCE LISTING (19 sequences)

SEQ ID NO: 1 - protein, 18 aa, Staphylococcus aureus
CYFSSKDNVGKVTGGKTC

SEQ ID NO: 2 - DNA, 54 bp, Staphylococcus aureus
tgctatttttcatccaaagataatgtaggtaaagttacaggtggcaaaacttgt

SEQ ID NO: 3 - protein, 14 aa, Artificial
  Synthetic
CYFSSKDNAGGKTC

SEQ ID NO: 4 - DNA, 42 bp, Artificial
  Synthetic
tgctatttttcatccaaagataatgcaggtggcaaaacttgt

SEQ ID NO: 5 - protein, 9 aa, Artificial
  Synthetic
CYFSSGKTC

SEQ ID NO: 6 - DNA, 27 bp, Artificial
  Synthetic
tgctatttttcatccggcaaaacttgt

SEQ ID NO: 7 - protein, 6 aa, Artificial
  Synthetic
CCGKTC

SEQ ID NO: 8 - DNA, 18 bp, Artificial
  Synthetic
tgctgtggcaaaacttgt

SEQ ID NO: 9 - protein, 240 aa, Staphylococcus aureus
  misc_feature (240)..(240): Xaa can be any naturally occurring amino acid
ESQPDPTPDELHKASKFTGLMENMKVLYDDHYVSATKVKSVDKFLAHDLIYNISDKKLKNYDKVKTELLNEGLAKKYKDEVVDVYGSNYYVNCYFSSKDNVGKVTGGKTCMYGGITKHEGNHFDNGNLQNVLIRVYENKRNTISFEVQTDKKSVTAQELDIKARNFLINKKNLYEFNSSPYETGYIKFIENNGNTFWYDMMPAPGDKFDQSKYLMMYNDNKTVDSKSVKIEVHLTTKNGX

SEQ ID NO: 10 - protein, 240 aa, Staphylococcus aureus
  misc_feature (240)..(240): Xaa can be any naturally occurring amino acid
ESQPDPTPDELHKSSEFTGTMGNMKYLYDDHYVSATKVMSVDKFLAHDLIYNISDKKLKNYDKVKTELLNEDLAKKYKDEVVDVYGSNYYVNCYFSSKDNVGKVTGGKTCMYGGITKHEGNHFDNGNLQNVLIRVYENKRNTISFEVQTDKKSVTAQELDIKARNFLINKKNLYEFNSSPYETGYIKFIENNGNTFQYDMMPAPGDKFDQSKYLMMYNDNKTVDSKSVKIEVHLTTKNGX

SEQ ID NO: 11 - protein, 240 aa, Staphylococcus aureus
  misc_feature (240)..(240): Xaa can be any naturally occurring amino acid
ESQPDPMPDDLHKSSEFTGTMGNMKYLYDDHYVSATKVKSVDKFLAHDLIYNISDKKLKNYDKVKTELLNEDLAKKYKDEVVDVYGSNYYVNCYFSSKDNVGKVTGGKTCMYGGITKHEGNHFDNGNLQNVLVRVYENKRNTISFEVQTDKKSVTAQELDIKARNFLINKKNLYEFNSSPYETGYIKFIENNGNTFQYDMMPAPGDKFDQSKYLMMYNDNKTVDSKSVKIEVHLTTKNGX

SEQ ID NO: 12 - protein, 240 aa, Staphylococcus aureus
  misc_feature (240)..(240): Xaa can be any naturally occurring amino acid
ESQPDPMPDDLHKSSEFTGTMGNMKYLYDDHYVSATKVKSVDKFLAHDLIYNINDKKLNNYDKVKTELLNEDLANKYKDEVVDVYGSNYYVNCYFSSKDNVGKVTSGKTCMYGGITKHEGNHFDNGNLQNVLIRVYENKRNTISFEVQTDKKSVTAQELDIKARNFLINKKNLYEFNSSPYETGYIKFIESNGNTFWYDMMPAPGDKFDQSKYLMIYKDNKMVDSKSVKIEVHLTTKNGX

SEQ ID NO: 13 - protein, 240 aa, Staphylococcus aureus
  misc_feature (240)..(240): Xaa can be any naturally occurring amino acid
ESQPDPTPDELHKSSEFTGTMGNMKYLYDDHYVSATKVKSVDKFLAHDLIYNISDKKLKNYDKVKTELLNEDLAKKYKDEVVDVYGSNYYVNCYFSSKDNVGKVTGGKTCMYGGITKHEGNHFDNGNLQNVLIRVYENKRNTISFEVQTDKKSVTAQELDIKARNFLINKKNLYEFNSSPYETGYIKFIENNGNTFQYDMMPAPGDKFDQSKYLMMYNDNKTVDSKRVKIEVHLTTKNGX

SEQ ID NO: 14 - protein, 240 aa, Staphylococcus aureus
  misc_feature (240)..(240): Xaa can be any naturally occurring amino acid
ESQPDPMPDDLHKSSEFTGTMGNMKYLYDDHYVSATKVKSVDKFLAHDLIYNISDKRLKNYDKVKTELLNEDLAKKYKDEVVDVYGSNYYVNCYFSSKDNVGKVTGGKTCMYGGITKHEGNHFDNGNLQNVLVRVYENKRNTISFEVQTDKKSVTAQELDIKARNFLINKKNLYEFNSSPYETGYIKFIENNGNTFQYDMMPAPGDKFDQSKYLMMYNDNKTVDSKRVKIEVHLTTKNGX

SEQ ID NO: 15 - protein, 240 aa, Staphylococcus aureus
  misc_feature (240)..(240): Xaa can be any naturally occurring amino acid
ESQPDPTPDELHKASKFTGLMENMKVLYDDRYVSATKVKSVDKFLAHDLIYNISDKKLKNYDKVKTELLNEDLAKKYKDEVVDVYGSNYYVNCYFFSKDNVGKVTGGKTCMYGGITKHEGNHFDNGNLQNVLIRVYENKRNTISFEVQTDKKSVTAQELDIKARNFLINKKNLYEFNSSPYETGYIKFIENNGNTFQYDMMPAPGDKFDQSKYLMMYNDNKTVDSKRVKIEVHLTTKNGX

SEQ ID NO: 16 - protein, 240 aa, Staphylococcus aureus
  misc_feature (240)..(240): Xaa can be any naturally occurring amino acid
ESQPDPTPDELHKASKFTGLMENMKVLYDDRYVSATKVKSVDKFLAHDLIYNISDKKLKNYDKVKTELLNEDLAKKYKDEVVDVYGSNYYVNCCFFSKDNVGKVTGGKTCMYGGITKHEGNHFDNGNLQNVLIRVYENKRNTISFEVQTDKKSVTAQELDIKARNFLINKKNLYEFNSSPYETGYIKFIENNGNTFQYDMMPAPGDKFDQSKYLMMYNDNKTVDSKRVKIEVHLTTKNGX

SEQ ID NO: 17 - protein, 228 aa, Artificial
  Synthetic
  misc_feature (228)..(228): Xaa can be any naturally occurring amino acid
ESQPDPTPDELHKASKFTGLMENMKVLYDDHYVSATKVKSVDKFLAHDLIYNISDKKLKNYDKVKTELLNEGLAKKYKDEVVDVYGSNYYVNCCGKTCMYGGITKHEGNHFDNGNLQNVLIRVYENKRNTISFEVQTDKKSVTAQELDIKARNFLINKKNLYEFNSSPYETGYIKFIENNGNTFWYDMMPAPGDKFDQSKYLMMYNDNKTVDSKSVKIEVHLTTKNGX

SEQ ID NO: 18 - protein, 231 aa, Artificial
  Synthetic
  misc_feature (231)..(231): Xaa can be any naturally occurring amino acid
ESQPDPTPDELHKASKFTGLMENMKVLYDDHYVSATKVKSVDKFLAHDLIYNISDKKLKNYDKVKTELLNEGLAKKYKDEVVDVYGSNYYVNCYFSSGKTCMYGGITKHEGNHFDNGNLQNVLIRVYENKRNTISFEVQTDKKSVTAQELDIKARNFLINKKNLYEFNSSPYETGYIKFIENNGNTFWYDMMPAPGDKFDQSKYLMMYNDNKTVDSKSVKIEVHLTTKNGX

SEQ ID NO: 19 - protein, 236 aa, Artificial
  Synthetic
  misc_feature (236)..(236): Xaa can be any naturally occurring amino acid
ESQPDPTPDELHKASKFTGLMENMKVLYDDHYVSATKVKSVDKFLAHDLIYNISDKKLKNYDKVKTELLNEGLAKKYKDEVVDVYGSNYYVNCYFSSKDNAGGKTCMYGGITKHEGNHFDNGNLQNVLIRVYENKRNTISFEVQTDKKSVTAQELDIKARNFLINKKNLYEFNSSPYETGYIKFIENNGNTFWYDMMPAPGDKFDQSKYLMMYNDNKTVDSKSVKIEVHLTTKNGX